(19) United States
(12) Patent Application Publication
(10) Pub. No.: US 2024/0041354 A1
(43) Pub. Date: Feb. 8, 2024

(54) TRACKING CALORIC EXPENDITURE USING A CAMERA

(71) Applicant: Apple Inc., Cupertino, CA (US)

(72) Inventors: Aditya Sarathy, Santa Clara, CA (US); James P. Ochs, San Francisco, CA (US); Yongyang Nie, New York, NY (US)

(21) Appl. No.: 18/357,089

(22) Filed: Jul. 21, 2023

Related U.S. Application Data
(60) Provisional application No. 63/394,905, filed on Aug. 3, 2022.

(51) Int. Cl. A61B 5/11; A61B 5/00; G06T 7/254; G06T 7/246; G06V 40/16

(52) U.S. Cl. CPC A61B 5/1118; A61B 5/4866; A61B 5/1123; A61B 5/0022; A61B 5/1128; G06T 7/254; G06T 7/248; G06V 40/171; G06T 2207/30201

(57) ABSTRACT

The enclosed embodiments are directed to tracking caloric expenditure using a camera. In an embodiment, a method comprises: obtaining face tracking data associated with a user; determining a step cadence of the user based on the face tracking data; determining a speed of the user based on the step cadence and a stride length of the user; obtaining device motion data from at least one motion sensor of the device; determining a grade of a surface on which the user is walking or running based on at least one of the device motion data or the face tracking data; and determining an energy expenditure of the user based on the estimated speed, the estimated grade and a caloric expenditure model.

### CROSS-REFERENCE TO RELATED APPLICATION

This application claims priority to U.S. Provisional Patent Application No. 63/394,905, filed Aug. 3, 2022, the entire contents of which is incorporated herein by reference.

### TECHNICAL FIELD

This disclosure relates generally to fitness applications.

### BACKGROUND

Tracking devices (e.g., smartphones and smartwatches), include motion sensors that are used by fitness applications to estimate the caloric expenditure of a user during physical activity. In some fitness applications, motion data from the motion sensors (e.g., acceleration, rotation rate) are used with an energy expenditure model to estimate the amount of calories burned by the user during a workout. Some applications also use a heart rate sensor in combination with the energy expenditure model to estimate calories burned. These fitness applications require that the tracking device be worn on the body of the user, which is typically the wrist, chest, torso or foot.

### SUMMARY

Embodiments are disclosed for tracking caloric expenditure using a camera.

In some embodiments, a method comprises: obtaining, with at least one processor of a device, face tracking data associated with a user; determining, with the at least one processor, a step cadence of the user based on the face tracking data; determining, with the at least one processor, a speed of the user based on the step cadence and a stride length of the user; obtaining, with the at least one processor, device motion data from at least one motion sensor of the device; determining, with the at least one processor, a grade of a surface on which the user is walking or running based on at least one of the device motion data or the face tracking data; and determining, with the at least one processor, an energy expenditure of the user based on the estimated speed, the estimated grade and a caloric expenditure model.

In some embodiments, the method further comprises: capturing, with a camera of the device, video data of the user's face; and generating, with the at least one processor, the face tracking data from the video data.

In some embodiments, the device is a mobile phone and the camera is a front-facing camera of the mobile phone.

In some embodiments, the method further comprises: correcting, with the at least one processor, the face tracking data to remove vertical face motion due to the user nodding their head.

In some embodiments, determining, with the at least one processor, the step cadence of the user based on the face tracking data further comprises: extracting features indicative of a step from a window of the face tracking data; and computing the step cadence based on the extracted features.

In some embodiments, the features include at least one of the following features: 1) one period in vertical displacement and a half a period of horizontal displacement; 2) one horizontal velocity cusp and vertical velocity cusp within each step; 3) the horizontal and vertical velocity intersect near a time of a foot strike where the user's foot is touching the ground; or 4) the horizontal velocity, vertical velocity and vertical displacement amplitudes exceed specified thresholds.

In some embodiments, determining the grade of the surface on which the user is walking or running based on at least one of the device motion data or the face tracking data, further comprises: tracking a displacement envelope of a vertical axis of a face centered reference frame; responsive to the envelope changing, estimating the grade of the surface based on the face tracking data; and responsive to the envelope not changing, determining the grade of the surface based on device motion data output by a motion sensor of the device.

In some embodiments, the method further comprises: computing, with the at least one processor, an uncalibrated stride length of the user based at least in part on a height of the user; computing, with the at least one processor, an uncalibrated distance by multiplying the step cadence and the uncalibrated stride length; computing, with the at least one processor, a calibration factor by dividing a truth distance by the uncalibrated distance, and then multiplying the uncalibrated stride length by the calibration factor to get a calibrated stride length; and computing, with the at least one processor, the speed of the user by multiplying the step cadence by the calibrated stride length.

In some embodiments, the truth distance is obtained or derived from a global navigation satellite system (GNSS) receiver.

In some embodiments, a system comprises: at least one processor; memory storing instructions that when executed by the at least one processor, cause the at least one processor to perform any of the preceding methods.

In some embodiments, a non-transitory, computer-readable storage medium having stored thereon instructions, that when executed by at least one processor, causes the at least one processor to perform any of the preceding methods.

Advantages of the disclosed embodiments include: 1) providing a real-time treadmill fitness experience using the front-facing camera of a tracking device (e.g., a smartphone) when the device is not worn by the user; 2) track caloric expenditure during the user's workout using speed and grade estimated using a single camera; and 3) no need for body-worn sensors (e.g., a smartwatch) or fitness machine connectivity.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIGS. 1A-1C illustrate coordinate reference frames tracking a user's face using a front-facing camera, according to an embodiment.

FIG. 2 illustrates key features of a step that can be extracted from face tracking data to estimate the user's step cadence, according to an embodiment.

FIGS. 3A and 3B illustrate face vertical axis position over time during a grade change for a treadmill design where the control/display console is detached from the running deck, according to an embodiment.

FIGS. 3C and 3D illustrate device motion pitch data over time during a grade change for a treadmill design where the control/display console is connected to the running deck, according to an embodiment.

FIG. 4 is block diagram of a system for estimating user step cadence and grade using face tracking data capture from video and device motion data, according to an embodiment.

FIG. 5 is a block diagram of a system for estimating caloric expenditure based on the estimated user step cadence and estimated grade, according to an embodiment.

FIG. 6 is a block diagram illustrating estimating user speed based on the estimated step user step cadence and calibrated user stride length, according to an embodiment.

FIG. 7 is a flow diagram of a process of tracking caloric expenditure using a camera, according to some embodiments.

FIG. 8 is example system architecture implementing the features and operations described in reference to FIGS. 1-7.

### DETAILED DESCRIPTION

### Example Speed Estimation

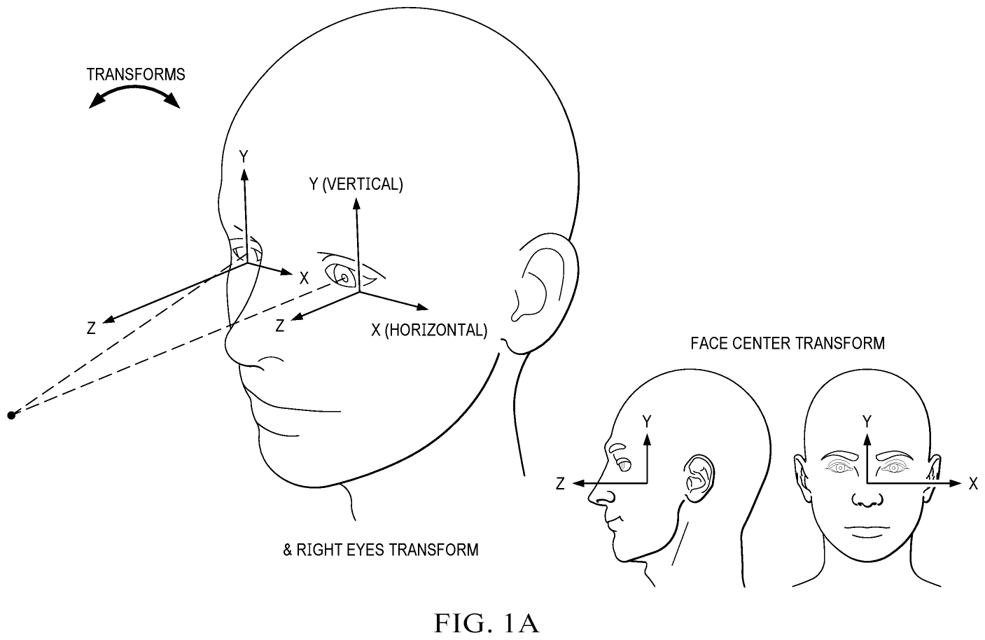

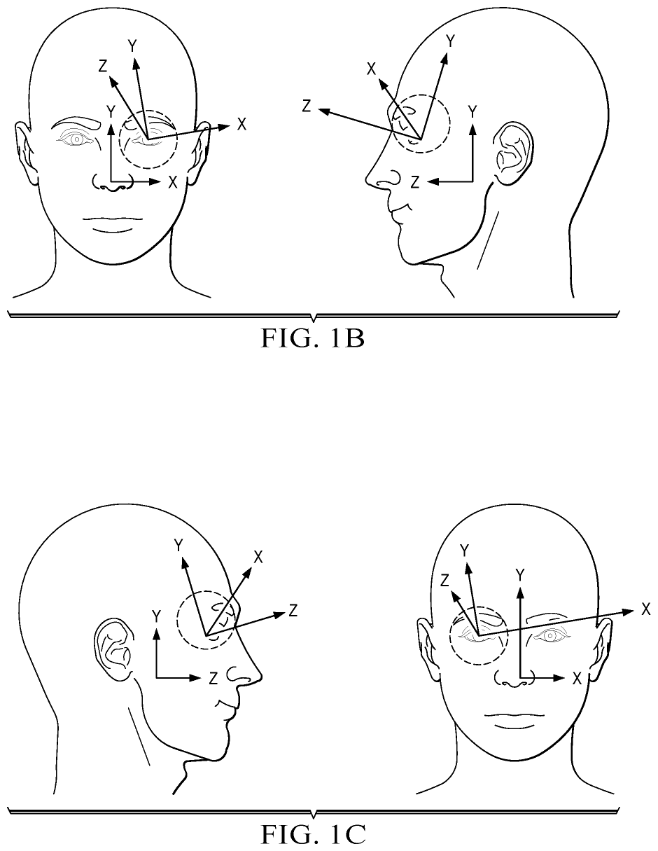

FIGS. 1A-1C illustrate coordinate reference frames tracking a user's face using a front-facing camera, according to an embodiment.

In some embodiments, a face tracker of a tracking device (e.g., face tracking software of a smartphone) receives video frames captured by a front-facing camera of the tracking device, such as a smartphone. The tracking device can be, for example, a smartphone resting on a tread mill console while a user is exercising on a treadmill. The face tracker uses various coordinate transformations to track the motion of one or more features of the user's face. In the embodiments that follow, the face tracker is part of Apple Inc.'s ARKit®, and the facial features tracked include the user's face center (e.g., the user's nose), left eye and right eye. Each feature is represented by a Cartesian reference frame. The face center reference frame is illustrated in FIG. 1A and the reference frames for the left eye and right eye balls are illustrated in FIGS. 1B and 1C, respectively. The translation aspect of these three matrices indicate the position of the center of the head/eyeballs, relative to a reference position represented by an anchor transform, where the anchor transform represents a real-world position and orientation of the user's face.

Referring to FIGS. 1A-1C, the positive z-axis points from the center of the face/eyeballs (in the direction of the nose and pupils, respectively). The rotational information of the matrices (first 3 columns of a 4×4 matrix) indicate the orientation of the face/eyeballs. For example, a rotation about the x-axis directs the head/pupil upward or downward. Note that the eyes do not rotate about the z-axis.

In addition, to the face center, left and right eye transforms, the face tracker also outputs a look-at point. The look-at-point is represented by a vector which is abstracted from the left and right eye transforms matrices to estimate what point, relative to the user's face, the user's eyes are focused upon. For example, if the user is looking to the left, the look-at point vector has a positive x-axis component, if the user is focused on a nearby object, the vector's length is shorter and if the user is focused on a faraway object, the vector's length is longer. As described in further detail below, the look-at point vector is used to correct head motion due to the user nodding their head while they exercise.

Although the embodiments described herein utilize Apple Inc.'s ARKit®, any suitable face tracking software can be used that provides motion data as described herein, or that can be mathematically manipulated to provide motion data as described herein. Some examples of face trackers include but are not limited to: Vuforia™, Unity™, ARCore™, Unreal Engine™, Blender™, Amazon Sumerian™, Kivy™, Godot™, HP Reveal™, ZapWorks™ and the like. In some embodiments, the output of the face tracker is a 4×4 transform matrix (e.g., a direction cosine matrix) indicating the position and orientation of the facial feature. In other embodiments, there can be more or fewer facial features tracked, different reference frames utilized and/or different output representations of position/orientation provided by the face trackers (e.g., quaternions rather than direction cosine matrices).

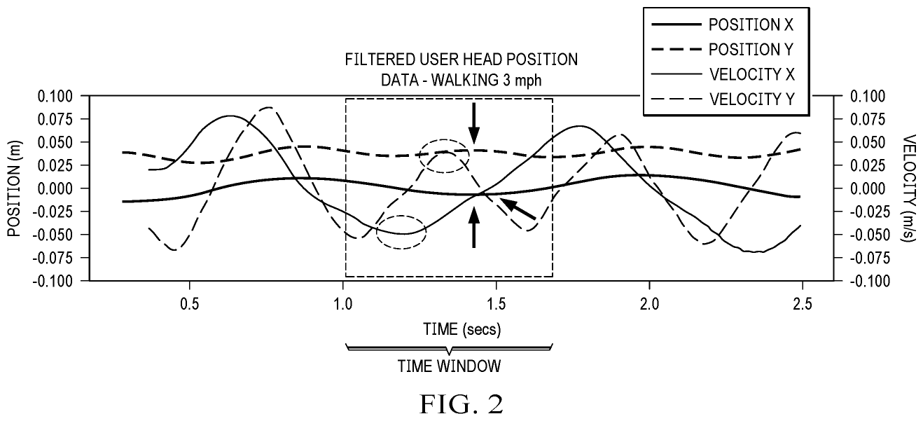

FIG. 2 illustrates key features of a step that can be extracted from face tracking data output by the face tracker to estimate the user's step cadence, according to an embodiment. In this example, a multivariate plot of filtered user head position (m) and velocity (m/s) versus time (secs) is shown for a user walking on a treadmill at 3 mph. An example time window (e.g., 1.70 secs) is also shown that captures certain key features that indicate that the user has taken a step.

The key features that indicate a step include: 1) one period in y (vertical) displacement and half a period of x (horizontal) displacement; 2) one x (horizontal) velocity cusp and y (vertical) velocity cusp within each step (see dashed ovals); 3) the x (horizontal) and y (vertical) velocity intersect near a time of a foot strike (i.e., the user's foot touching the ground); and 4) the x (horizontal), y (vertical) velocity and y (vertical) displacement amplitudes exceed specified thresholds. If all four of these key features are present in a single time window, then a step is detected. In some embodiments, more or fewer key features can be used to indicate a step. The step detections taken over a unit of time (e.g., per sec) indicate the user's step cadence, which can be multiplied by the user's stride length to estimate the user's speed, as described in further detail below. As used in this example embodiment, the x (horizontal) axis and the y (vertical) axis form a face-centered Cartesian coordinate system as shown in FIG. 1A, where the x or horizontal axis points in the direction of the user's left ear, the y or vertical axis points out of the top of the user's head and the z axis points along the user's nose based on the right-hand rule.

### Example Grade Estimation

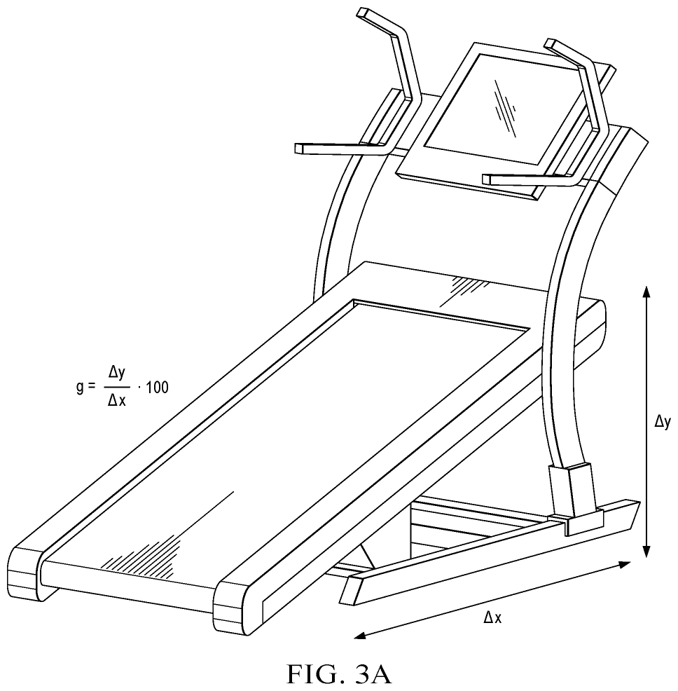

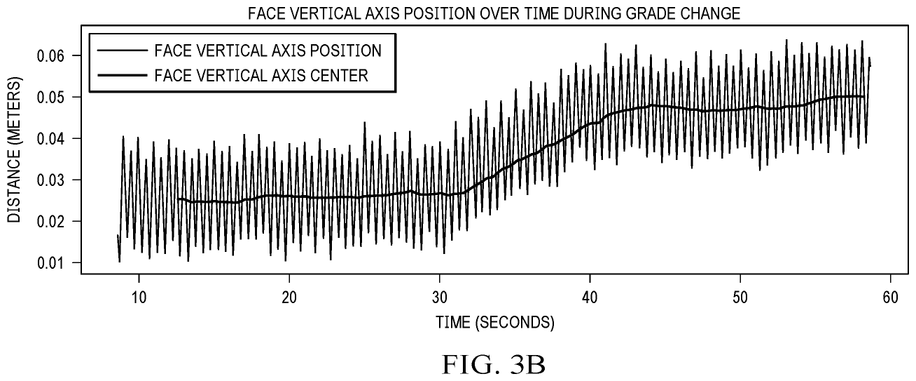

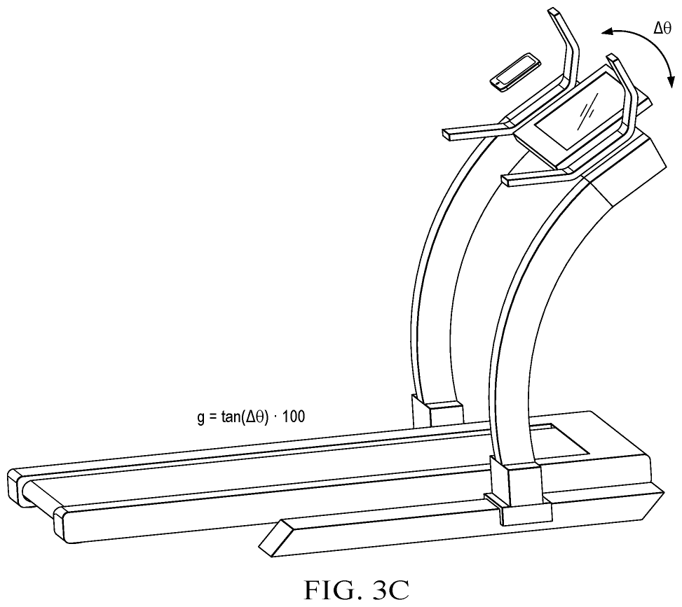

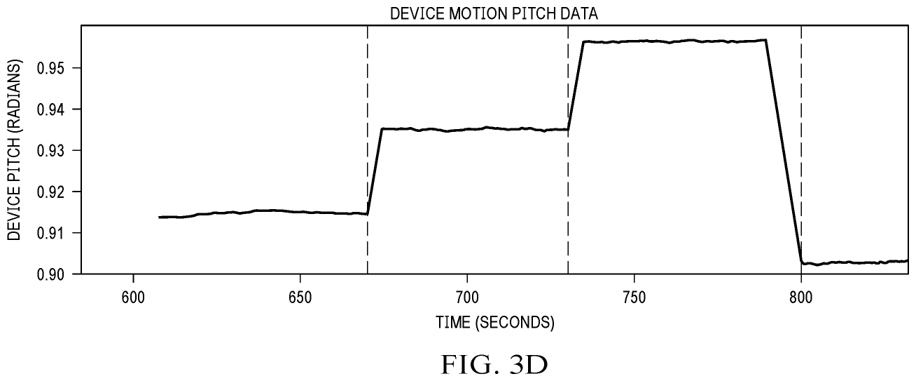

FIGS. 3A and 3B illustrate a first treadmill design where the treadmill console is stationary when the incline of the running deck is changed. For this first treadmill design, grade is computed as:

$$g = \frac{Δ y}{Δ X} , \qquad [ 1 ]$$

where Δy is the vertical displacement of the running deck from ground and Δx is horizontal displacement of the running deck, as illustrated in FIG. 3A. As shown in FIG. 3B, grade is estimated based on a detected change of the vertical axis (y-axis) center of the face-centered reference frame over time. Note that the displacement envelope of the face vertical axis center changes when the incline/grade of the running deck changes. In this example, at about the 30 second mark a grade change causes the face vertical axis center to transition upwards until about the forty second mark where the grade change ends. In some embodiments, face vertical axis center is provided by the face tracker, and is corrected for head nodding/bobbing by subtracting the look-at point vector (described above) from the face vertical axis center.

FIGS. 3C and 3D illustrate device motion pitch change over time during a grade change for a treadmill design where the control/display console is connected to the running deck, according to an embodiment. For this treadmill design, grade g is computed as:

g=tan(Δθ)*100, [2]

where Δθ is the pitch of the treadmill console relative to ground.

In some embodiments, device motion data is used as an approximation of Δθ. For example, a gyro sensor in the tracking device outputs pitch data (Δθ), which is used to estimate grade according to Equation [2].

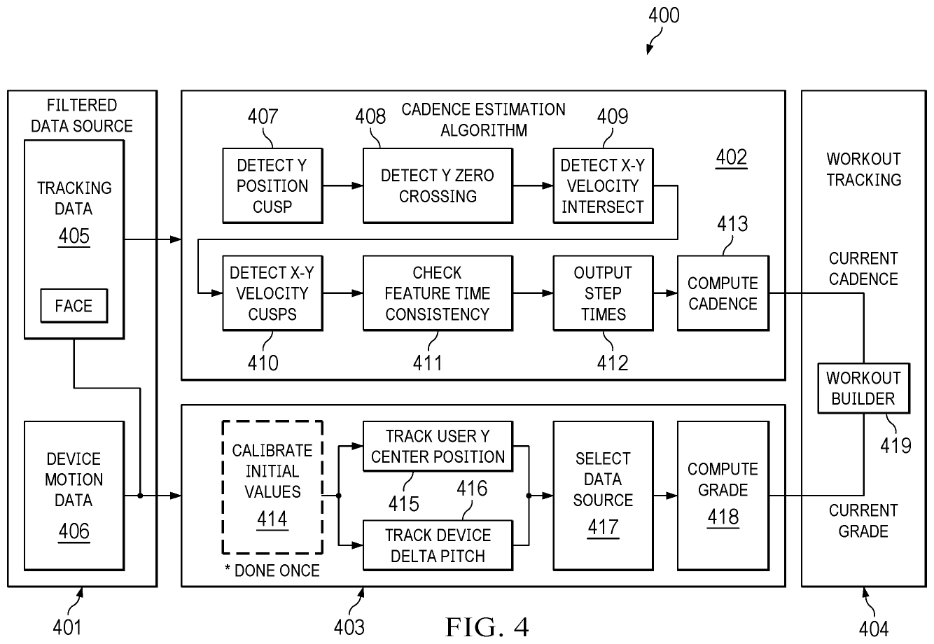

FIG. 4 is block diagram of a system for estimating user step cadence and grade using face tracking data and device motion data, according to an embodiment. System 400 includes data filtering 401, cadence estimator 402, grade estimator 403 and workout tracker 404.

Data filtering 401 filters face tracking data and device motion data. Filtering can include but is not limited to windowing and filtering the face tracking data and device motion data to remove noise. The filtering can include, for example, averaging the tracking data and device motion data to remove noise.

Cadence estimator 402 estimates the user's step cadence by detecting key features in the face tracking data provided by a face tracker within a specified time window, as shown in FIG. 2. The key feature detecting includes detecting x-y position cusps 407, detecting y crossing 408 (indicating a foot strike), detecting x-y velocity intersection 409 (near the y crossing 408), detecting x-y velocity cusps 410, checking feature time consistency 411 (checking that there is one period in y displacement and a half period of x displacement in a single window), outputting user's step times 412 and computing user's step cadence 413 based on the user's step times.

Grade estimator 403 estimates grade from device motion. At initialization, initial values are calibrated 414. Calibration can include but is not limited to generating reference transforms based on a current position and orientation of the tracking device. After initialization, the vertical axis position of the user's face 415 from the face tracking data (i.e., the y component of the x, y, z position vector provided in the face tracking data) and device motion pitch 416 are employed to detect grade change, as described in reference to FIGS. 3A, 3B. In step 417, a particular grade model (e.g., Equation [1] or Equation [2]) is selected to estimate grade. For example, if the face vertical axis center is changing and the device motion pitch is not changing, then Equation [1] is used to compute grade. Similarly, if the face vertical axis center is not changing and the device motion pitch is changing, then Equation [2] is used to compute grade. If both the face vertical axis center and device motion pitch are changing, it is assumed that the tracking device is not stationary on the console (e.g., it is being handled by the user) and no grade estimate is computed.

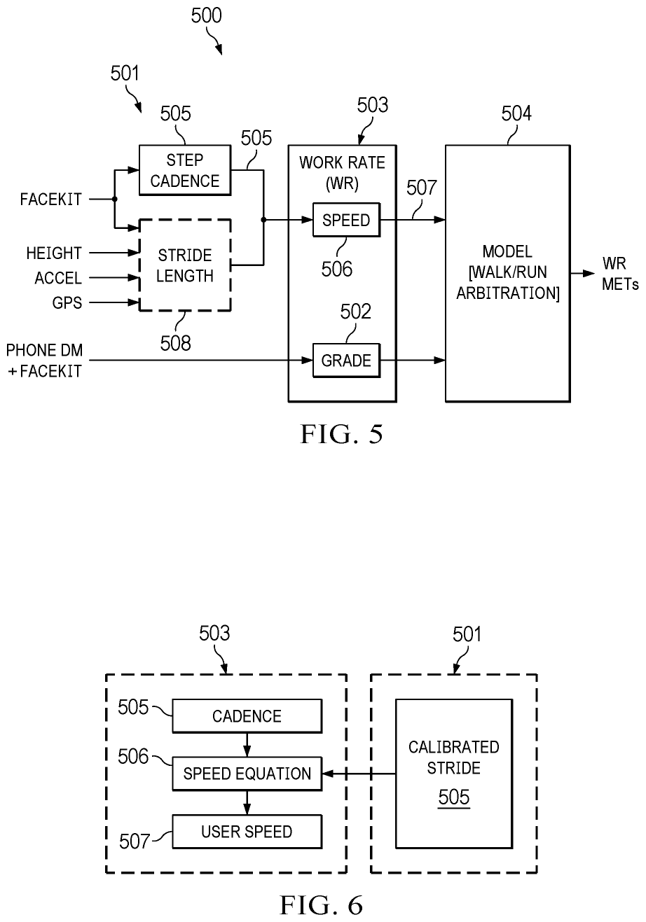

In some embodiments, workout tracker 404 receives the current step cadence of the user and the current grade computed by cadence estimator 402 and grade estimator 403, and computes estimated work rate (WR) metabolic equivalent for task (METs), as described in reference to FIG. 5.

FIG. 5 is a block diagram of a system 500 for estimating caloric expenditure based on the estimated user step cadence and estimated grade, according to an embodiment. System 500 includes stride length estimator 501 and WR generator 503 (which comprises speed calculator 506 and grade calculator 502) and WR model 504. The output of the face tracker is the user's step cadence an as described in reference to FIG. 4. Stride length estimator 510 takes as input the user's step cadence, height, acceleration and speed. Height can be provided by the user through a user interface of the tracking device or otherwise accessed from a storage device. Acceleration can be provided by an accelerometer of the tracking device and speed can be provided by a global navigation satellite system (GNSS) receiver, such as a global positioning system (GPS) receiver embedded in or coupled to the tracking device.

In some embodiments, an uncalibrated stride length (or average stride length) is computed by multiplying the user's height by a ratio that accounts for the user's gender (e.g., 0.413 for women and 0.415 for men). An uncalibrated distance is then calculated by multiplying the step cadence output by the face tracker and the uncalibrated stride length. While the user is on the treadmill, a calibration factor k is calculated by dividing a “truth” distance taken from GNSS position from a wearable device (e.g., a smart watch) by the uncalibrated distance, and then multiplying the uncalibrated stride length by the calibration factor k to get a calibrated stride length 508 shown in FIG. 6. Speed calculator 506 includes speed equation 506 which multiplies the user's step cadence 505 output by the face tracker by the calibrated stride length 508 to get the user's speed 507, also shown in FIG. 6. Grade calculator 502 uses at least one of face tracking data or device motion (e.g., pitch from a gyro) to determine a surface grade depending on the type of treadmill that the user is exercising on, as described in reference to FIGS. 3A and 3B.

The estimated speed and grade is input into a WR energy expenditure model 504 which estimates the caloric expenditure of the user in METs or any other desirable unit that describes caloric expenditure. WR energy expenditure model 504 can include a model for aerobic capacity (VO2) for walking and for running. For example, an American College of Sports Medicine (ACSM) energy expenditure model for walking is given by:

VO2=(0.1*S)+(1.8*S*G)+C, [3]

where S is the estimated user speed, G is the estimated grade and C is a resting component (e.g., 3.5). Similarly, the ACSM energy expenditure model for running is given by:

VO2=(0.2*S)+(0.9*S*G)+C, [4]

where S is the estimated user speed and G is the estimated grade and C is a resting component (e.g., 3.5).

One MET equates to 3.5 ml·kg−1·min−1. Therefore dividing VO2 Max by the one MET value converts VO2 Max into METs.

These energy expenditure models are only examples of possible model that can benefit from the disclosed embodiments. Those with ordinary skill in the art will recognize that the disclosed embodiments can be used with any energy expenditure model that utilizes user speed and/or grade, including but not limited to any energy expenditure models based on work rate or that combine work rate based energy expenditure with heart rate based energy expenditure. Also, the disclosed embodiments are not limited to treadmill workouts, but can be used with any exercise or exercise equipment where camera tracking data provides an indication of a fitness metric, including but not limited to tracking other parts of the human body. For example, the disclosed embodiments are applicable to any exercise, health monitoring application or fitness machine where vertical or lateral face motion can be captured by a camera, including but not limited to counting squats, pull-ups or jump rope steps, where there is vertical motion of the face, or lateral motion of the face, such as side step exercises.

### Exemplary Process

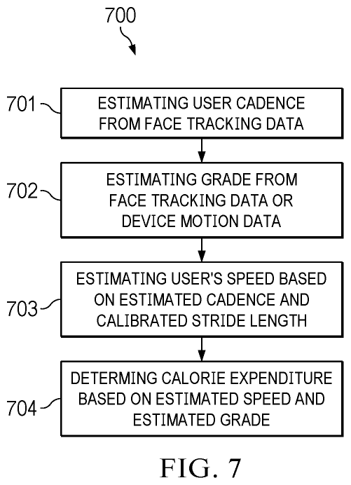

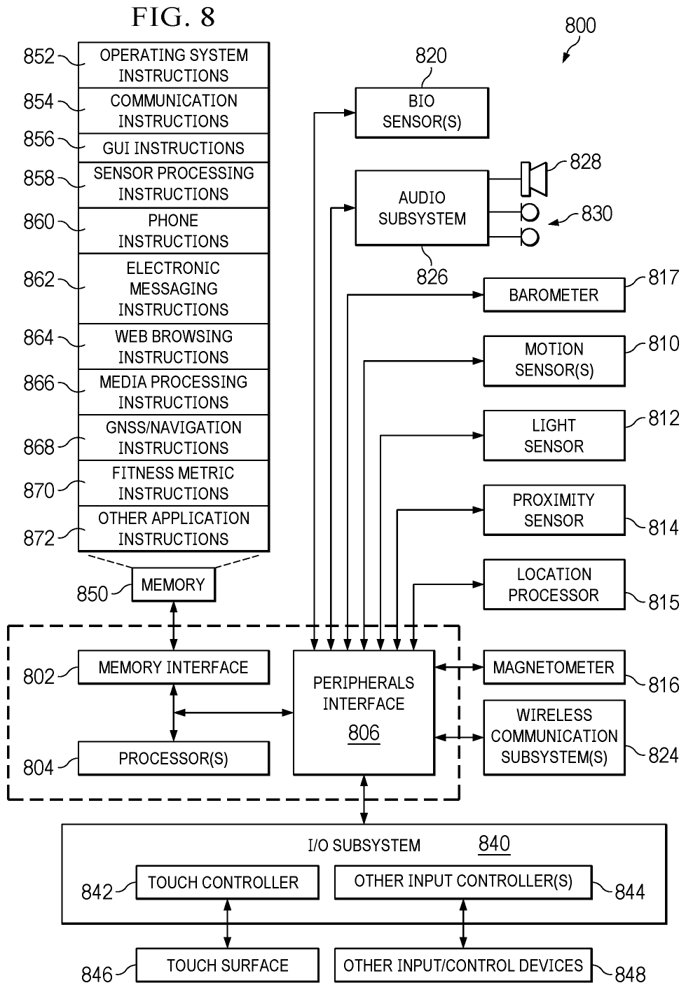

FIG. 7 is a flow diagram of process 700 of tracking caloric expenditure using a camera, according to an embodiment. Process 700 can be implemented by, for example, the device architecture 800 described in reference to FIG. 8.

Process 700 includes obtaining, from a camera of a tracking device, face tracking data associated with a user (701), determining a step cadence of the user based on the face tracking data (702), estimating a speed of the user based on the step cadence and a stride length of the user (703), obtaining device motion data from at least one motion sensor of the device (704), estimating a grade of a surface on which the user is walking or running based on at least one of the device motion data or the face tracking data (705), and estimating, using a caloric expenditure model, an energy expenditure of the user based on the estimated speed and estimated grade (706).

### Exemplary System Architecture

FIG. 8 illustrates example device architecture 800 implementing the features and operations described in reference to FIGS. 1-7. Architecture 800 can include memory interface 802, one or more hardware data processors, image processors and/or processors 804 and peripherals interface 806. Memory interface 802, one or more processors 804 and/or peripherals interface 806 can be separate components or can be integrated in one or more integrated circuits. System architecture 800 can be included in any suitable electronic device, including but not limited to: a smartphone, smartwatch, tablet computer, fitness band, laptop computer and the like.

Sensors, devices and subsystems can be coupled to peripherals interface 806 to provide multiple functionalities. For example, one or more motion sensors 810, light sensor 812 and proximity sensor 814 can be coupled to peripherals interface 806 to facilitate motion sensing (e.g., acceleration, rotation rates), lighting and proximity functions of the tracking device. Location processor 818 can be connected to peripherals interface 806 to provide geo-positioning. In some implementations, location processor 815 can be a GNSS receiver, such as the Global Positioning System (GPS) receiver. Electronic magnetometer 816 (e.g., an integrated circuit chip) can also be connected to peripherals interface 806 to provide data that can be used to determine the direction of magnetic North. Electronic magnetometer 816 can provide data to an electronic compass application. Motion sensor(s) 810 can include one or more accelerometers and/or gyros configured to determine change of speed and direction of movement. Barometer 817 can be configured to measure atmospheric pressure, which can be used to determine elevation changes.

Communication functions can be facilitated through wireless communication subsystems 824, which can include radio frequency (RF) receivers and transmitters (or transceivers) and/or optical (e.g., infrared) receivers and transmitters. The specific design and implementation of the communication subsystem 824 can depend on the communication network(s) over which a tracking device is intended to operate. For example, architecture 800 can include communication subsystems 824 designed to operate over a GSM network, a GPRS network, an EDGE network, a Wi-Fi™ network and a Bluetooth™ network. In particular, the wireless communication subsystems 824 can include hosting protocols, such that the tracking device can be configured as a base station for other wireless devices.

Audio subsystem 826 can be coupled to a speaker 828 and a microphone 830 to facilitate voice-enabled functions, such as voice recognition, voice replication, digital recording and telephony functions. Audio subsystem 826 can be configured to receive voice commands from the user.

I/O subsystem 840 can include touch surface controller 842 and/or other input controller(s) 844. Touch surface controller 842 can be coupled to a touch surface 846. Touch surface 846 and touch surface controller 842 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 846. Touch surface 846 can include, for example, a touch screen or the digital crown of a smart watch. I/O subsystem 840 can include a haptic engine or device for providing haptic feedback (e.g., vibration) in response to commands from one or more processors 804. In an embodiment, touch surface 846 can be a pressure-sensitive surface.

Other input controller(s) 844 can be coupled to other input/control devices 848, such as one or more buttons, rocker switches, thumb-wheel, infrared port and USB port. The one or more buttons (not shown) can include an up/down button for volume control of speaker 828 and/or microphone 840. Touch surface 846 or other controllers 844 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 846; and a pressing of the button for a second duration that is longer than the first duration may turn power to the tracking device on or off. The user may be able to customize a functionality of one or more of the buttons. The touch surface 846 can, for example, also be used to implement virtual or soft buttons.

In some implementations, the tracking device can present recorded audio and/or video files, such as MP3, AAC and MPEG files. In some implementations, the tracking device can include the functionality of an MP3 player. Other input/output and control devices can also be used.

Memory interface 802 can be coupled to memory 850. Memory 850 can include high-speed random access memory and/or non-volatile memory, such as one or more magnetic disk storage devices, one or more optical storage devices and/or flash memory (e.g., NAND, NOR). Memory 850 can store operating system 852, such as the iOS operating system developed by Apple Inc. of Cupertino, California. Operating system 852 may include instructions for handling basic system services and for performing hardware dependent tasks. In some implementations, operating system 852 can include a kernel (e.g., UNIX kernel).

Memory 850 may also store communication instructions 854 to facilitate communicating with one or more additional devices, one or more computers and/or one or more servers, such as, for example, instructions for implementing a software stack for wired or wireless communications with other devices, such as a sleep/wake tracking device. Memory 850 may include graphical user interface instructions 856 to facilitate graphic user interface processing; sensor processing instructions 858 to facilitate sensor-related processing and functions; phone instructions 860 to facilitate phone-related processes and functions; electronic messaging instructions 862 to facilitate electronic-messaging related processes and functions; web browsing instructions 864 to facilitate web browsing-related processes and functions; media processing instructions 866 to facilitate media processing-related processes and functions; GNSS/Location instructions 868 to facilitate generic GNSS and location-related processes and instructions; and caloric expenditure instructions 870 that implement the features and processes described in reference to FIGS. 1-7. Memory 850 further includes other application instructions 872 for performing various functions using, for example, health monitoring or fitness applications.

Each of the above identified instructions and applications can correspond to a set of instructions for performing one or more functions described above. These instructions need not be implemented as separate software programs, procedures, or modules. Memory 850 can include additional instructions or fewer instructions. Furthermore, various functions of the tracking device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a tracking device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor of a device, face tracking data associated with a user;
determining, with the at least one processor, a step cadence of the user based on the face tracking data;
determining, with the at least one processor, a speed of the user based on the step cadence and a stride length of the user;
obtaining, with the at least one processor, device motion data from at least one motion sensor of the device;
determining, with the at least one processor, a grade of a surface on which the user is walking or running based on at least one of the device motion data or the face tracking data; and
determining, with the at least one processor, an energy expenditure of the user based on the estimated speed, the estimated grade and a caloric expenditure model.

2. The method of claim 1, further comprising:
capturing, with a camera of the device, video data of the user's face; and
generating, with the at least one processor, the face tracking data from the video data.

3. The method of claim 2, wherein the device is a mobile phone and the camera is a front-facing camera of the mobile phone.

4. The method of claim 1, further comprising:
correcting, with the at least one processor, the face tracking data to remove vertical face motion due to the user nodding their head.

5. The method of claim 1, wherein determining, with the at least one processor, the step cadence of the user based on the face tracking data further comprises:
extracting features indicative of a step from a window of the face tracking data; and
computing the step cadence based on the extracted features.

6. The method of claim 5, wherein the features include at least one of the following features: 1) one period in vertical displacement and a half a period of horizontal displacement; 2) one horizontal velocity cusp and vertical velocity cusp within each step; 3) the horizontal and vertical velocity intersect near a time of a foot strike where the user's foot is touching the ground; or 4) the horizontal velocity, vertical velocity and vertical displacement amplitudes exceed specified thresholds.

7. The method of claim 1, wherein determining the grade of the surface on which the user is walking or running based on at least one of the device motion data or the face tracking data, further comprises:
tracking a displacement envelope of a vertical axis of a face centered reference frame;
responsive to the envelope changing, estimating the grade of the surface based on the face tracking data; and
responsive to the envelope not changing, determining the grade of the surface based on device motion data output by a motion sensor of the device.

8. The method of claim 1, further comprising:
computing, with the at least one processor, an uncalibrated stride length of the user based at least in part on a height of the user;
computing, with the at least one processor, an uncalibrated distance by multiplying the step cadence and the uncalibrated stride length;
computing, with the at least one processor, a calibration factor by dividing a truth distance by the uncalibrated distance, and then multiplying the uncalibrated stride length by the calibration factor to get a calibrated stride length; and
computing, with the at least one processor, the speed of the user by multiplying the step cadence by the calibrated stride length.

9. The method of claim 8, wherein the truth distance is obtained or derived from a global navigation satellite system (GNSS) receiver.

10. A system comprising:
at least one processor;
memory storing instructions that when executed by the at least one processor, cause the at least one processor to perform operations comprising:
obtaining face tracking data associated with a user;
determining a step cadence of the user based on the face tracking data;
determining a speed of the user based on the step cadence and a stride length of the user;
obtaining device motion data from at least one motion sensor of the device;
determining a grade of a surface on which the user is walking or running based on at least one of the device motion data or the face tracking data; and
determining an energy expenditure of the user based on the estimated speed, the estimated grade and a caloric expenditure model.

11. The system of claim 10, wherein the operations further comprise:
capturing, with a camera of the device, video data of the user's face; and
generating, with the at least one processor, the face tracking data from the video data.

12. The system of claim 11, wherein the device is a mobile phone and the camera is a front-facing camera of the mobile phone.

13. The system of claim 10, wherein the operations further comprise:
correcting, with the at least one processor, the face tracking data to remove face motion caused by the user nodding their head.

14. The system of claim 10, wherein determining the step cadence of the user based on the face tracking data further comprises:
extracting features indicative of a step from a window of the face tracking data; and
computing the step cadence based on the extracted features.

15. The system of claim 14, wherein the features include at least one of the following features: 1) one period in vertical displacement and a half a period of horizontal displacement; 2) one horizontal velocity cusp and vertical velocity cusp within each step; 3) the horizontal and vertical velocity intersect near a time of a foot strike where the user's foot is touching the ground; or 4) the horizontal velocity, vertical velocity and vertical displacement amplitudes exceed specified thresholds.

16. The system of claim 10, wherein determining the grade of the surface on which the user is walking or running based on at least one of the device motion data or the face tracking data, further comprises:
tracking a displacement envelope of a vertical axis of a face centered reference frame;
responsive to the envelope changing, estimating the grade of the surface based on the face tracking data; and
responsive to the envelope not changing, determining the grade of the surface based on device motion data output by a motion sensor of the device.

17. The system of claim 10, wherein the operations further comprise:
computing an uncalibrated stride length of the user based at least in part on a height of the user;
computing an uncalibrated distance by multiplying the step cadence and the uncalibrated stride length;
computing a calibration factor by dividing a truth distance by the uncalibrated distance, and then multiplying the uncalibrated stride length by the calibration factor to get a calibrated stride length; and
computing the speed of the user by multiplying the step cadence by the calibrated stride length.

18. The system of claim 17, wherein the truth distance is obtained or derived from a global navigation satellite system (GNSS) receiver.

19. A non-transitory, computer-readable storage medium having stored thereon instructions, that when executed by at least one processor, causes the at least one processor to perform operations comprising:
obtaining face tracking data associated with a user;
determining a step cadence of the user based on the face tracking data;
determining a speed of the user based on the step cadence and a stride length of the user;
obtaining device motion data from at least one motion sensor of the device;
determining a grade of a surface on which the user is walking or running based on at least one of the device motion data or the face tracking data; and
determining an energy expenditure of the user based on the estimated speed, the estimated grade and a caloric expenditure model.

20. The non-transitory, computer-readable storage medium of claim 19, wherein determining the step cadence of the user based on the face tracking data further comprises:
extracting features indicative of a step from a window of the face tracking data; and
computing the step cadence based on the extracted features.
